(19) United States
(12) Patent Application Publication
(10) Pub. No.: US 2008/0221388 A1
(43) Pub. Date: Sep. 11, 2008

(54) SIDE VIEWING OPTICAL FIBER ENDOSCOPE

(75) Inventors: Eric J. Seibel, Seattle, WA (US); Richard S. Johnston, Sammamish, WA (US); Charles David Melville, Issaquah, WA (US); Janet L. Crossman-Bosworth, Kenmore, WA (US)

Correspondence Address: LAW OFFICES OF RONALD M ANDERSON, 600 108TH AVE, NE, SUITE 507, BELLEVUE, WA, 98004, US

(73) Assignee: University of Washington, Seattle, WA (US)

(21) Appl. No.: 11/684,417

(22) Filed: Mar. 9, 2007

(51) Int. Cl. A61B 1/07

(52) U.S. Cl. 600/109

(57) ABSTRACT

An optical fiber conveys light from a source at a proximal end, to a distal end, where a piezoelectric material tube applies a force that causes the distal end of the optical fiber to scan in a desired pattern. Light from the distal end of the optical fiber passes through a lens system and is at least partially reflected by a reflective surface toward a side of the scope, to illuminate tissue within a patient's body. Light received from the internal tissue is reflected back either to collection optical fibers, which convey the light to proximally disposed optical detectors, or directly toward distal optical detectors. The optical detectors produce electrical signals indicative of an intensity of the light that can be used for producing an image of the internal tissue. The light received from the tissue can be either scattered, polarized, fluorescent, or filtered, depending on the illumination light.

### GOVERNMENT RIGHTS

This invention was funded at least in part with a grant (No. CA094303) from the National Institutes of Health/National Cancer Institute (NIH/NCI), and the U.S. government may have certain rights in this invention.

### BACKGROUND

Endoscopes and other types of scopes used for imaging and collecting optical data of internal sites within a body of a patient typically image in a forward direction, i.e., downstream of the distal end of the scope. However, side-viewing scopes are also sometimes used because they are capable of viewing internal conditions within a body lumen or cavity, to the side of the scope.

For either forward viewing or side-viewing, the resolution of a conventional scope is limited by the imaging device used to produce images. Small diameter scopes are particularly desirable for introduction into body lumens or spaces that are also relatively small. As the cross-sectional size of a scope becomes smaller, the resolution of a conventional scope also usually decreases, since the number of optical fibers that can be bundled together is limited by the reduced size of the scope.

For example, one application of a small diameter scope is to carry out a diagnostic evaluation of a patient's pancreatic duct. A scope that is suitable for this task should be less than 3 mm in diameter, have a rigid distal tip that is ≦20 mm in length, and be capable of producing a high frequency and high amplitude scan that can provide an undistorted two-dimensional (2-D) scanned focal plane at high optical or spatial resolution. The scope should also have side-viewing capability meeting these optical criteria, to enable the walls of the pancreatic duct to be evaluated as the scope is advanced through the relatively small diameter duct. While a forward viewing scope can also image and enable diagnostic evaluation of a small diameter body lumen, a side-viewing scope can more effectively be used for this purpose, since the imaging and other optical evaluation is less distorted and can be implemented without requiring imaging with as great a depth of focus range. However, conventional side-viewing scopes are much too large and fail to provide the necessary resolution to achieve acceptable results in very small diameter body lumens.

It would also be desirable to evaluate tissue within a body lumen of a patient's body using a side-viewing scope that is able to image using light having different polarization characteristics. The scope should also be capable of detecting scattered, or fluorescent light received from tissue at the side of the scope. Other desirable features of a side-viewing scope include low cost, relatively high flexibility to enable a scope to be readily advanced through tortuous passages with relatively sharp turns within a patient's body, and the ability to provide pixel-accurate delivery of diagnostic and therapeutic optical radiation to an internal site proximate the distal end of the scope.

### SUMMARY

An exemplary side-viewing scope for imaging a region inside a body of a patient has been developed and is described in detail below. The side-viewing scope includes an optical fiber that extends between a proximal end and a distal end. The proximal end of the optical fiber is configured to couple to an external light source to receive light produced by the external light source and to convey the light toward the distal end of the optical fiber for use in illuminating a region disposed proximate to the distal end of the optical fiber. A scanning device is disposed at the distal end of the optical fiber and is coupled thereto. The scanning device has a free end from which light conveyed through the optical fiber is emitted in a first direction. An actuator is included for providing a driving force to move the free end of the scanning device in a desired pattern. A reflective surface is disposed adjacent to the free end of the scanning device and reflects at least a portion of the light emitted from the free end in a second direction that is generally transverse to the first direction, so that the portion of the light reflected from the reflective surface is directed towards a side of the scope. At least one light detector is provided for detecting light from a region disposed at a side of the scope illuminated by the light reflected from the reflective surface. The one or more light detectors produce a signal that is usable to produce an image of the region.

The reflective surface can be one of four different options. These options include: (1) a mirror that reflects the light emitted by the scanning device in the second direction; (2) a triangular element having two opposite faces that are reflective and which reflect the light emitted by the scanning device in opposite directions, either of the opposite directions comprising the second direction and the other of the opposite directions comprising a third direction; (3) an axially-symmetric mirror surface or a cone having a reflective surface, or a pyramidal element having more than two faces that are reflective, each reflecting light emitted by the scanning device in a different direction towards the side of the scope; or, (4) a partially-reflective beamsplitter that reflects a portion of the light emitted by the scanning device towards the side of the scope and transmits the remainder.

In at least one exemplary embodiment, the partially-reflective beamsplitter transmits a remaining portion of the light emitted by the scanning device towards the distal end of the scope to illuminate another region. This other region is disposed forward of and proximate to the distal end of the scope, enabling forward viewing by the scope.

The side-viewing scope can further include at least one collection optical fiber having a proximal end and a distal end. The reflective surface also reflects light received from the side of the scope back into the distal end of the at least one collection optical fiber for transmission toward the proximal end of the at least one collection optical fiber.

In at least one exemplary embodiment, the proximal end of each collection optical fiber is configured to couple to a corresponding light detector that detects at least one specific type of light. The specific types of light can be either parallel polarized light, perpendicularly polarized light, scattered light that has been scattered from tissue, fluorescent light emitted by tissue, or filtered light backscattered from the tissue.

For an alternative exemplary embodiment that does not include a collection optical fiber, one or more light detectors are disposed adjacent to the distal end of the scanning device for receiving light from tissue disposed at the side of the scope, and in at least another exemplary embodiment, also from tissue disposed beyond the distal tip of the scope. The signal produced by the light detector(s) corresponds to an intensity of the light that is received from the tissue.

In at least one exemplary embodiment, the side-viewing scope also includes electrical leads that have a distal end and a proximal end. The distal ends of the electrical leads are connected to the at least one light detector for conveying each signal produced thereby to the proximal end of the leads, for coupling to a processing device.

The actuator is configured to apply a driving force to the free end of the scanning device, causing the free end to move at about its resonant frequency. This actuator can cause the scanning device to move in the desired pattern to implement one of several different types of scans. These different types of scans include a linear scan, a raster scan, a sinusoidal scan, a toroidal scan, a spiral scan, and a propeller scan.

Another aspect of this new development is directed to a method for imaging a region disposed at a side of a distal end of a scope that is configured to be introduced into a patient's body. The steps of the method are generally consistent with functions carried out by the components of the side-viewing scope discussed above.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is a schematic diagram of a possible lens system design for an optical fiber scanning system, which allows both a forward and a side view;

FIG. 2A is a schematic diagram of another lens design for an optical fiber scanning system, which provides a side view;

FIG. 2B is a schematic diagram of a lens design for an optical fiber scanning system, which provides a side view in a similar fashion to FIG. 2A but with a larger spacing between the lenses in order to move the image plane closer to the endoscope;

FIG. 3 is a schematic diagram of a lens design for an optical fiber scanning system, which provides both a forward and side view by virtue of its very wide field of view;

FIG. 4A is a schematic diagram of an arrangement for two reflective surfaces, which would provide side views for an optical fiber scanning system;

FIG. 4B is a schematic diagram of a pyramidal arrangement for four reflective surfaces, which would provide side views for an optical fiber scanning system;

FIG. 5 is a schematic diagram of an axially-symmetric reflective surface in an optical fiber scanning system, which would have significant image distortion but could accurately distinguish such general tissue conditions as color and fluorescence at an axial position of the scope within a body lumen;

FIG. 6A is a schematic diagram of an annular detector ring which includes six optical detectors and six lead wires coming from the detectors, in an optical fiber scanning system;

FIG. 6B is a schematic diagram of an annular detector ring which includes six multimode fibers for conveyance of light in an optical fiber scanning system;

FIG. 7A is a schematic diagram of the scanning mechanism and the single mode fiber to be scanned, in an optical fiber scanning system;

FIG. 7B is a schematic diagram of a single mode fiber with a microlens at the distal tip, vibrating in second mode resonance;

FIG. 8 is an exemplary block diagram illustrating the functional flow of signals in a system that is usable with an optical fiber for imaging, monitoring, and rendering diagnoses, in accord with the present invention;

FIG. 9 is a schematic diagram of the part of an optical fiber scanning system, which would be used internally in a patient, and which illustrates the use of a single mirror and collection optical fibers for collecting light from an internal site;

FIG. 10 illustrates an exemplary embodiment of a scope that includes a beamsplitter to provide both side and forward viewing and includes annular rings of optical detectors for detecting light from internal tissue disposed at both the side and distally of the scope;

FIG. 11 is a schematic diagram of another exemplary embodiment of a scope used in an optical fiber scanning system, illustrating the use of an axially-symmetric reflective conical surface for scanning tissue at an internal site;

FIG. 12A is a schematic diagram of yet another exemplary embodiment of a scope that uses a mirror assembly having two or more reflective surfaces;

FIG. 12B is a schematic diagram of an alternative exemplary embodiment of a scope that is similar to that of FIG. 12A, but which uses an annular detector ring instead of multimode collection fibers for detecting light from tissue at an internal site; and

FIG. 13 is a schematic diagram of an exemplary wire grid polarizer, which can be used in the detection of polarized light in an optical fiber scanning system when placed over the distal end of collection optical fibers that are disposed in an annular ring around the scanning optical fiber, such as in the scope shown in FIG. 12A.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

### Exemplary Side-Viewing Optical Fiber Scanning Systems

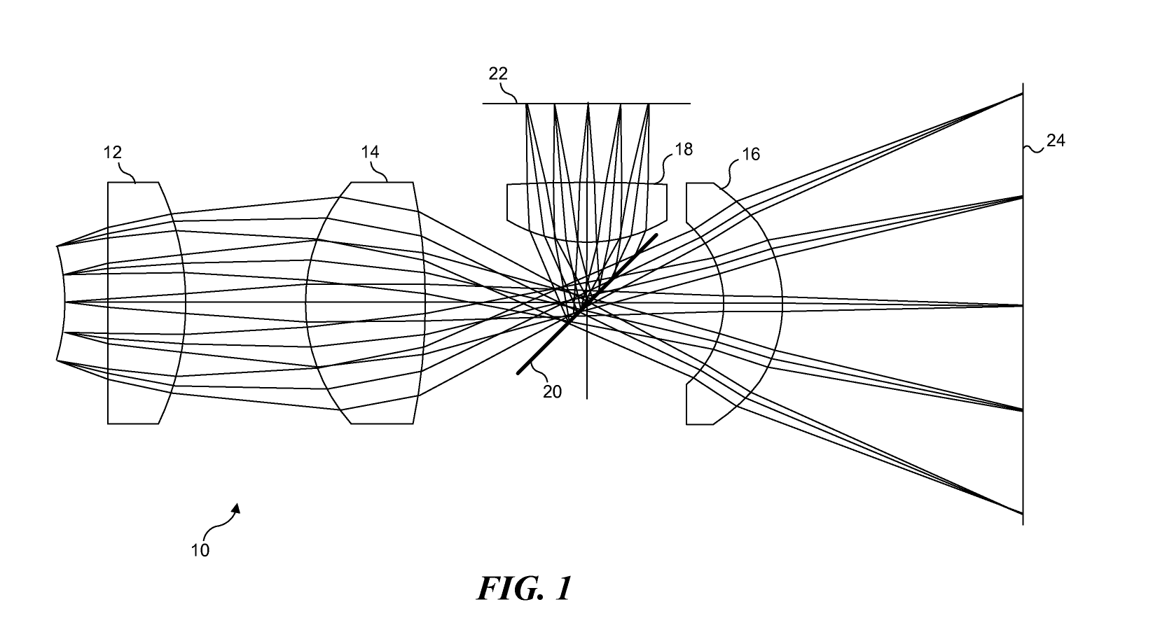

Referring to FIG. 1, a lens system 10 for use in a side-viewing optical fiber scanning system includes four lenses 12, 14, 16, and 18, and a forty-five degree beamsplitter 20. Beamsplitter 20 can be a partially-reflective mirror, a dichroic beamsplitter, or a polarizing beamsplitter. Lens system 10 provides both a forward and a side view of a region of interest (ROI) disposed inside the body of a patient. This ROI may be the tissue inside a body lumen, such as an esophagus, a bile duct, a pancreatic duct, a lung airway, or other such tubular organ (none shown). Forty-five degree beamsplitter 20 is configured so that certain wavelengths of light that are scanned by the side-view optical fiber scanning system are reflected towards the side of the lens system, and other wavelengths are transmitted through the dichroic beamsplitter towards the end of the lens system. Three lenses 12, 14, and 16, which provide the forward view, are 3.0 mm in diameter in this exemplary embodiment, and lens 18 is 2.0 mm in diameter and in combination with lenses 12 and 14 and dichroic beamsplitter 20, provides a side view of the body lumen or duct. The estimated spatial resolution of the side view at image plane 22, calculated at a wavelength of 635 nm, is 10 microns. The estimated spatial resolution of the forward view at image plane 24, calculated at a wavelength of 670 nm, is 21 microns. The side view of image plane 22 has a 1.5 mm diameter field of view (FOV), and the forward view of image plane 24 has a 5.2 mm diameter FOV. This exemplary design is able to image inside an esophagus, which has an inner diameter of about 25 mm, or inside a bile duct, which has an inner diameter of about 5 mm. Modifications made to this design would enable it to fit into a smaller body lumen, such as a pancreatic duct, which has an inner diameter down to about 2 mm.

If beamsplitter 20 is not wavelength or polarization selective, it reflects a portion of the light transmitted through lens 14 to the side, toward image plane 22, while transmitting a remaining portion of that light through lens 16 toward image plane 24. If beamsplitter 20 is of the polarizing type, it will reflect linearly polarized light that is polarized in a first direction and transmit linearly polarized light that is polarized in a second direction that is orthogonal to the first direction.

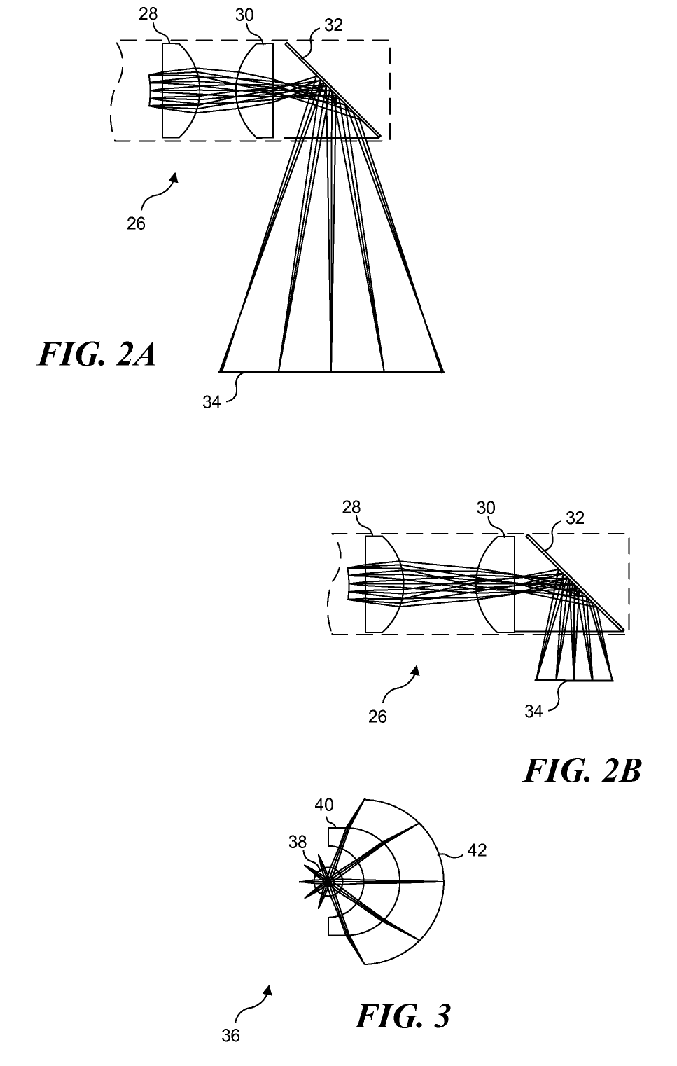

FIGS. 2A and 2B show an exemplary embodiment of another lens system 26 for a side-viewing optical fiber scanning system. A reflecting surface 32 (e.g., a front silvered mirror) disposed beyond lenses 28 and 30 provides an image plane 34 that is oriented at a 90 degree angle relative to the optical axis of the lens system (which extends through the centers of lenses 28 and 30). The greater spacing between lenses 28 and 30 in FIG. 2B (i.e., greater compared to the corresponding spacing shown in FIG. 2A) results in an image plane 34, which is closer to reflecting surface 32 than it is in FIG. 2A. The closer image plane shown in FIG. 2B enables viewing a ROI along the side of smaller diameter lumens of the body, as well as more greatly magnifying an area of tissue that is of interest. The lens system of FIG. 2A has an estimated spatial resolution of 27 microns, calculated at a wavelength of 635 nm, and image plane 34 in this exemplary embodiment has a 4.7 mm diameter FOV. The lens design of FIG. 2B has an estimated spatial resolution of about 9 microns, also calculated at a wavelength of 635 nm, and image plane 34 in FIG. 2B has about a 1.6 mm diameter FOV.

FIG. 3 shows an embodiment of a lens system 36 for a side-viewing optical fiber scanning system, which provides both a side view as well as a forward view. Due to its wide scan field, lens system 36 covers a FOV of 135 degrees. The scanned beam of light directed through a ball microlens 38 and a lens 40 provide the wide FOV at a curved image plane 42. The spatial resolution of this exemplary embodiment is estimated to be about 13 microns. Not shown is the optical fiber used for light delivery at a larger angular deflection, and with an effective point source disposed within the optical fiber at a position that is proximal of the ball microlens. Microlens 38 would be fused or otherwise coupled to this optical fiber.

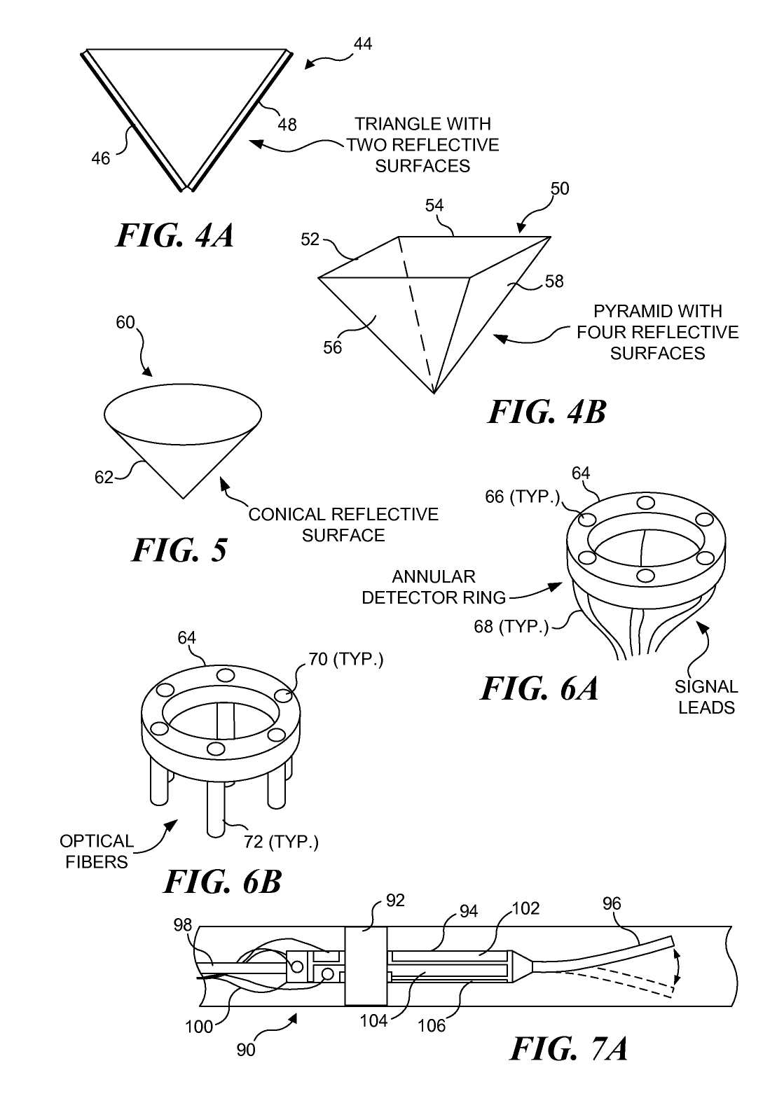

FIG. 4A shows a side view of an arrangement 44 of two mirrors 46 and 48, which can be positioned adjacent to the lens system of a side-viewing optical fiber scanning system in order to achieve two side views, in directions that are 180 degrees apart, e.g., toward opposite sides of a body lumen. The entire side-viewing optical fiber scanning system can be rotated during insertion and/or during a withdrawal of an endoscope (not shown in this Figure) that includes this arrangement of mirrors, so the entire lumen wall can be imaged while the endoscope is inserted or withdrawn, or the entire assembly can be rotated to image around a full 360 degrees in a body lumen at a stationary position in a body lumen. Rotating the field of view within the lumen simply requires rotating the entire shaft at the proximal end. Optionally, mirrors 46 and 48 can be rotated together by a small shaft coupled to a prime mover (not shown), so that the mirrors rotate around a common longitudinal axis that extends through the center of the line along which they contact each other. As they are thus rotated, mirrors 46 and 48 will enable viewing all 360 degrees of the peripheral side view. The same approach can be used to view a larger angular area or a full 360 degrees around the inner surface of a lumen, in connection with an exemplary embodiment of the side-viewing optical fiber scanning system that includes a single mirror, such as shown in FIGS. 2A and 2B.

FIG. 4B shows an exemplary pyramidal arrangement 50 of four mirrors 52, 54, 56, and 58, which can be disposed adjacent to the lens system in the scope of a side-viewing optical fiber scanning system in order to achieve four side views in directions that are oriented 90 degrees apart. These mirrors could also be rotated on a central shaft that extends through their common center vertex by a prime mover (not shown) in order to enable viewing of a full 360 degrees around the peripheral side view.

FIG. 5 shows an alternative exemplary conical embodiment 60 for viewing perpendicularly to the optical axis of a side-viewing optical fiber scanning system, using an axially-symmetric conical reflective surface 62. This conical reflective surface can provide a continuous 360 degree view of the periphery around a scope; however, since conical reflective surface 62 is not flat, the image it produces will suffer from significant distortion, and more specifically, a large amount of astigmatism. The advantage of the conical reflective surface is that it enables the system to accurately distinguish such general tissue conditions as color and fluorescence within a body lumen, which may be sufficient, if shape details of a region of interest are not particularly important.

FIG. 6A shows an exemplary annular ring 64 in which are mounted six optical detectors 66, such as photodiodes. Optical detectors 66 can be embedded in the front surface of the annular ring or otherwise affixed to it. Annular ring 64 can be used in a side-viewing optical fiber scanning system in order to detect light received from a patient's internal tissue disposed at the side of a scope (as well as forward of the scope in some exemplary embodiments, as discussed below), to enable the tissue to be imaged. Signal leads 68 are coupled to optical detectors 66 for transmitting the signal produced by each detector in response to incident light received from the internal site by the optical detectors, so that signal is conveyed to the proximal end of the system.

FIG. 6B illustrates annular ring 64 with the distal ends of six multimode collection optical fibers 70 embedded into the annular ring (instead of the optical detectors shown in FIG. 6A). The distal ends of these collection optical fibers are thus exposed on the upper surface of annular ring 64 to receive light from a patient's internal tissue that is being imaged with a side-viewing optical fiber scanning system, as discussed in greater detail below. Collection optical fibers 72 extend proximally below the annular ring in order to return the light that was received to detectors disposed more proximally in the side-viewing optical fiber scanning system. For example, the optical detectors (not shown) can be disposed externally of the patient's body, adjacent to the proximal end of the side-viewing optical fiber scanning system, or alternatively, can be disposed at an intermediate position.

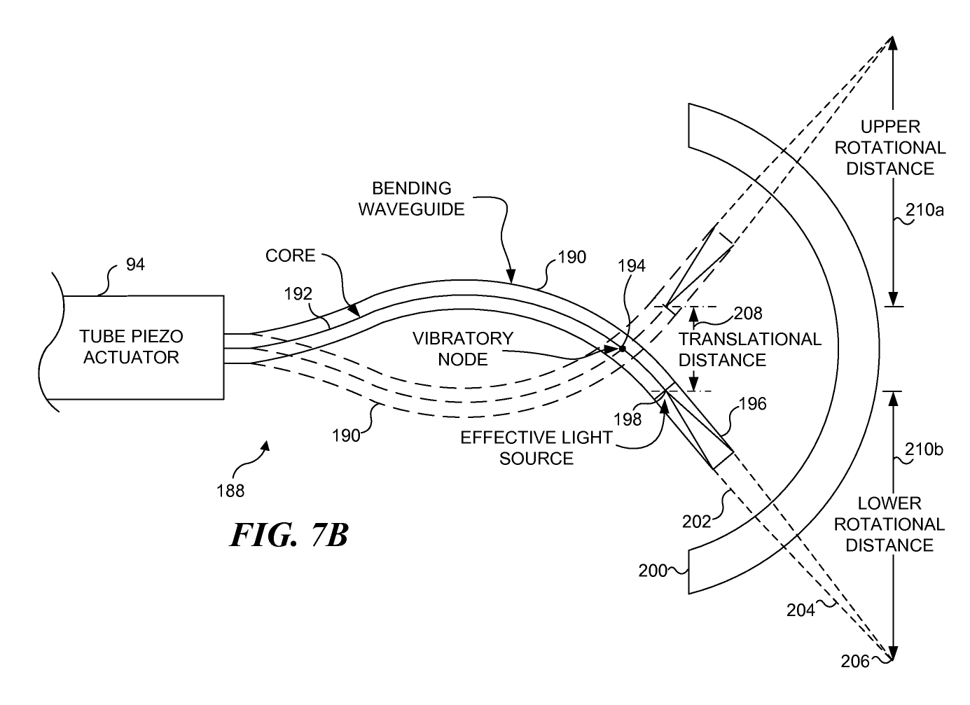

FIG. 7A shows an exemplary scanning mechanism 90 of a side-viewing optical fiber scanning system. Scanning mechanism 90 comprises a single mode optical fiber 98 that is supported by a tube 94 of piezoelectric material, which serves to drive a distal end 96 of the optical fiber to move in a desired scanning pattern. Distal end 96 extends distally beyond the tube of piezoelectric material and is cantilevered from it, generally within the center of the scope and adjacent to its distal end. This tube of piezoelectric material is held in the scanning apparatus by a base 92. Quadrant electrodes 102, 104, and 106 (and one other that is not visible in this view) are plated onto the tube of piezoelectric material and can be selectively energized with an applied voltage in order to generate two axes of motion in distal end 96 of optical fiber 98. Lead wires 100 carry electrical voltage signals to each of the quadrant electrodes to energize the piezoelectric material relative to each axis of motion. In this exemplary embodiment, the two axes are generally orthogonal to each other. An amplified sine wave applied to one axis and a cosine wave applied to the other axis of the tube piezoelectric material generate a circular scan, although those of ordinary skill in the art will understand that a variety of different scan patterns can be produced by appropriately moving distal end 96 of optical fiber 98. An appropriate modulation of the amplitudes of the electrical voltage signals applied to the quadrant electrodes can create a desired area-filling two dimensional pattern for imaging with light emitted from distal end 96 of the optical fiber. A few examples of the various scan patterns that can be achieved include a linear scan, a raster scan, a sinusoidal scan, a toroidal scan, a spiral scan, and a propeller scan. In some exemplary embodiments, the distal end is driven so that it moves at about a resonant (or near-resonant) frequency of the cantilevered distal end of optical fiber 98, which enables a greater amplitude to be achieved for the given drive signals applied.

FIG. 7A shows the first mode of lateral vibratory resonance of the cantilevered distal end of the optical fiber, whereas an optical fiber scanner 188 shown in FIG. 7B is being driven so that a cantilevered optical fiber 190 moves in the second mode of vibratory resonance. In FIG. 7B, the second node is disposed at about the distal end of cantilevered optical fiber 190 where it is fused to a microlens 196. In the exemplary embodiment of FIG. 7B, tube 94 piezoelectric material (or another suitable actuator) is again employed to excite cantilevered optical fiber 190 to move in a desired pattern, at a desired amplitude, and at a desired frequency. The dash lines show the corresponding shape and disposition of the cantilevered optical fiber when it is displaced 180 degrees in phase. Light is conveyed through a core 192 of cantilevered optical fiber 190 toward its distal end, where microlens 196 is attached. Microlens 196 can comprise a drum (barrel) lens, a gradient index (GRIN) lens, or a diffractive optical element. In this exemplary embodiment, when thus excited, the cantilevered optical fiber has a vibratory node 194 that is substantially proximal of an effective light source 198. Because of the displacement of vibratory node 194 from effective light source 198, scanning occurs primarily due to the rotation of microlens 196, but also due to the translation of the microlens and of the distal end of cantilevered optical fiber 190. Light 202 emitted from microlens 196 is slightly convergent and is focused by a scan lens 200 to produce focused light 204 that converges to a focal point 206. Movement of cantilevered optical fiber 190 thus causes effective light source 198 to move through a translation distance 208 and rotates the focused light generally through an upper rotational distance 210a and a lower rotational distance 210b (neither to scale). The scanning of focal point 206 for optical fiber scanner 188 thus results primarily from the rotation of the microlens, but also to a lesser extent, from the translation of the effective light source. As explained herein, the light emitted by the microlens can be directed to one or more sides of a side-viewing optical fiber scope to illuminate a ROI, for example, the interior surface tissue of a body lumen, as well as forward of the scope in some embodiments.

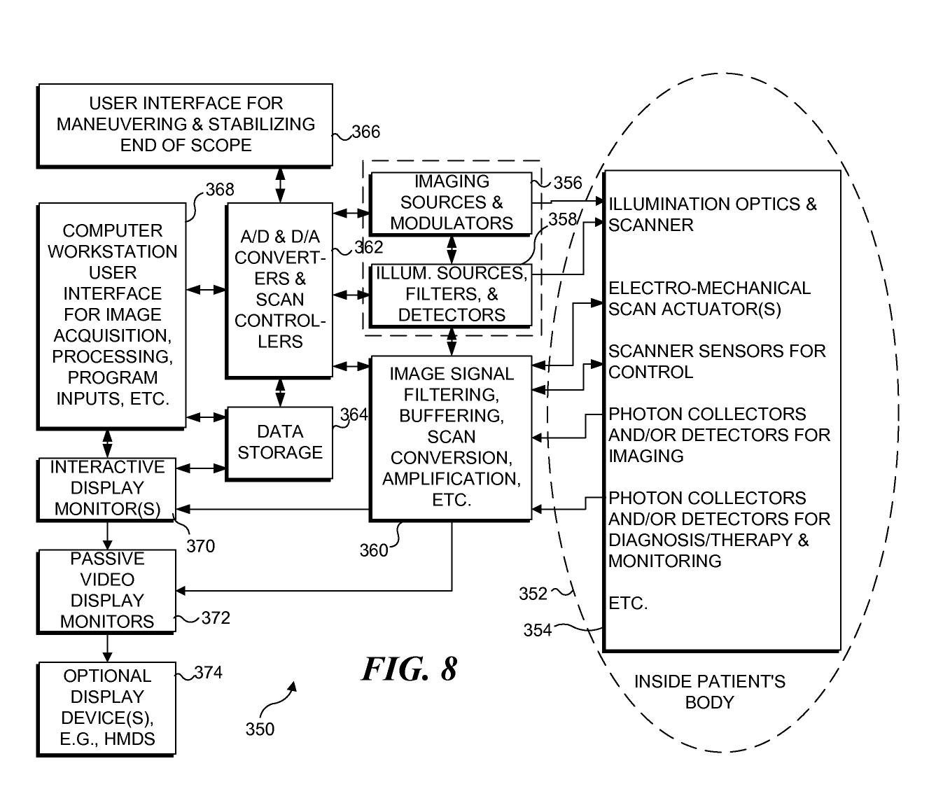

FIG. 8 illustrates a system 350 that shows how the signals produced by various components of a side-viewing scope that are inside a patient's body are processed with external instrumentation and how signals used for controlling the system to vary the scanning parameter(s) in successive scanning frames are input to the components that are positioned inside the patient's body on the side-viewing scanning optical fiber system. In order to provide integrated imaging and other functionality, system 350 is thus divided into the components that remain external to the patient's body, and those which are used internally (i.e., the components within a dash line 352). A block 354 lists the functional components disposed that can be included at the distal end of the side-viewing scanning optical fiber system (note that not all of these components are actually required for a side-viewing scanning optical fiber system). As indicated therein, these exemplary components can include illumination optics and scanner, one or more electromechanical scan actuator(s), one or more scanner sensors for control, photon collectors and/or detectors for imaging the desired site, and optionally, additional photon collectors and/or detectors for diagnostic purposes and for therapy and monitoring purposes—one or more of which can be implemented using the same scanning device by varying the illumination and/or scanning parameters that are employed during different scanning frames. It should again be noted that in regard to system 350, only the functional components actually required for a specific application may be included in a specific embodiment. Also, the additional functions besides imaging can be diagnostic, or therapy, or a combination of these functions.

Externally, the illumination optics and scanner(s) are supplied light from imaging sources and modulators, as shown in a block 356. Further details concerning several exemplary embodiments of external light source systems for producing RGB, UV, IR, polarized, and/or high intensity light conveyed to the distal end of an optical fiber system will be evident to a person of ordinary skill in this art. Scanner sensors can be used for controlling the scanning and can produce a signal that is fed back to the scanner actuators, illumination source, and modulators to implement scanning control after signal processing in a block 360. The sensors may simply be one or more temperature sensors, since temperature affects resonance and an open feedback system, based on initialization. Also, a temperature rise may occur due to the higher power therapy illumination transmitted through the system or indirectly as the result of thermal heat radiated back from the tissue. In many applications, scanner sensors will not be needed and can be omitted.

In block 360, image signal filtering, buffering, scan conversion, amplification, as well as other processing functions can be implemented using the electronic signals produced by the imaging photon collectors and/or detectors and by the other photon collectors and/or detectors employed for diagnosis/therapy, and monitoring purposes. Blocks 356 and 360 are interconnected bi-directionally to convey signals that facilitate the functions performed by each respective block. Similarly, each of these blocks is bi-directionally coupled in communication with a block 362 in which analog-to-digital (A/D) and digital-to-analog (D/A) converters are provided for processing signals that are coupled with a computer workstation user interface or other computing device employed for image acquisition, processing, for executing related programs, and for carrying out other useful functions. Control signals from the computer workstation are fed back to block 362 and converted into analog signals, where appropriate, for controlling or actuating each of the functions provided in blocks 356, 358, and 360. The A/D converters and D/A converters within block 362 are also coupled bi-directionally to a block 364 in which data storage is provided, and to a block 366. Block 366 represents a user interface for assisting in maneuvering, positioning, and stabilizing the end of the side-viewing scanning optical fiber endoscope within a patient's body.

In block 364, the data storage is used for storing the image data produced by the detectors within a patient's body, and for storing other data related to the imaging and functions implemented by the scanning optical fiber. Block 364 is also coupled bi-directionally to the computer workstation 368 and to interactive display monitor(s) in a block 370. Block 370 receives an input from block 360, enabling images of the ROI to be displayed interactively. In addition, one or more passive video display monitors may be included within the system, as indicated in a block 372. Other types of display devices, for example, a head-mounted display (HMD) system, can also be provided, enabling medical personnel to view a ROI as a pseudo-stereo image.

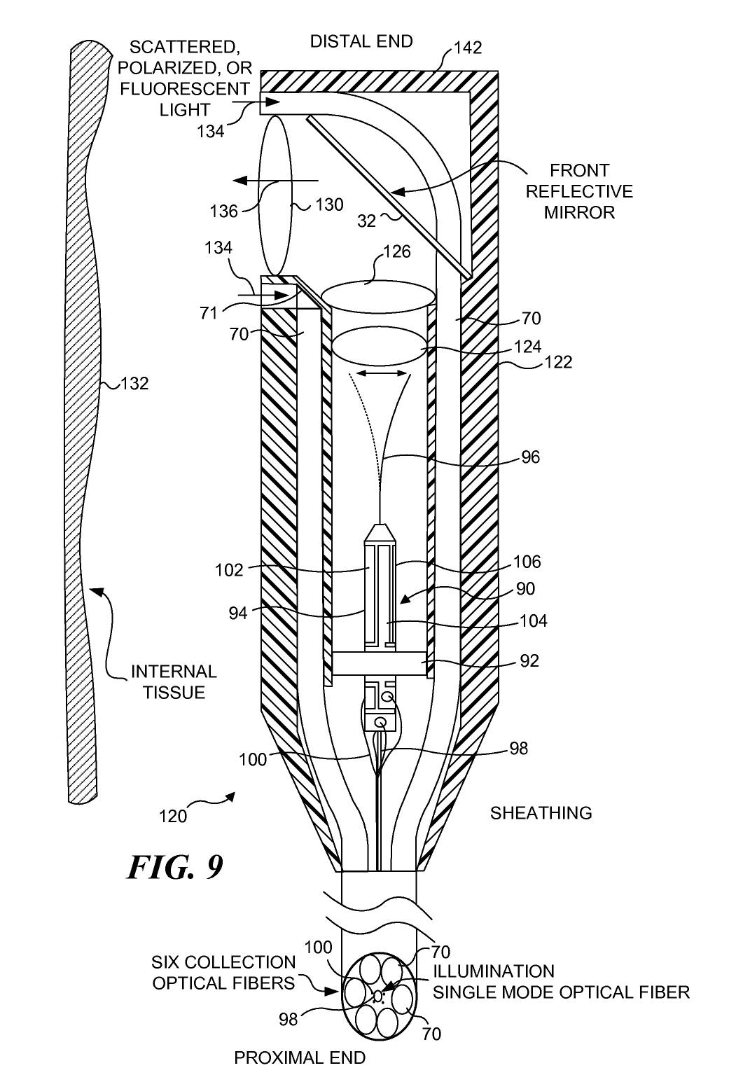

FIG. 9 is a schematic diagram of the distal part of an exemplary side-viewing optical fiber scanning system 120, which can be used internally in a patient, and which includes reflective surface 32 in order to image internal tissue at one side of the distal end of the system. (Note that the Figure is not drawn to scale and is much smaller in all dimensions, particularly in regard to the diameter of the illustrated scope.) Reflective surface 32 comprises a front-reflective mirror in this exemplary embodiment, in order to eliminate undesirable reflections, which would result if the back surface were coated with a reflective material, and light had to travel through a thickness of glass before reflecting from the reflective coating on the back surface. An end cap 142 supports reflective surface 32 and lens 130 in this exemplary embodiment.

Light from the proximal end of the system is directed through illumination single mode optical fiber 98 and travels in the optical fiber to the distal end of the system, which can be advanced to a desired internal site within a patient's body. Distal end 96 of the single mode optical fiber is vibrated in a desired scan pattern, e.g., a spiral pattern, by tube 94 of piezoelectric material, which is driven by electrical signals applied to its quadrant electrodes, as discussed above (in connection with FIG. 7A). The light exiting the moving distal end of the optical fiber travels through the lens system and is reflected outwardly by the reflective surface. Light 136 exiting from the side of the distal end of the system impacts internal tissue 132 of the patient, which might be disposed, for example, at the side of a body lumen.

Some of the light reflected by the tissue is collected by six collection optical fibers 70, the distal ends of which are generally disposed at spaced-apart locations around lens 130. Only two of the collection optical fibers are shown at the distal end of the system in the scope in FIG. 9, although all six are schematically illustrated at the proximal end. Where an abrupt change of direction is necessary for any of the collection optical fibers near lens 130, a mirror 71 (or other optical component such as a prism that exhibits total internal reflection (TIR) of the light) can be used to deflect the returning light into the adjacent distal end of one of the collection optical fibers. The light received from the internal tissue is conveyed along the collection optical fibers from the distal end of the system to its proximal end. Sheathing 122 supports the entire assembly at the distal end of the side-viewing optical fiber scanning system and facilitates its insertion into a lumen or other internal site of the patient. Optical fiber scanning system 120 can be used to collect scattered light, polarized light, or fluorescent light from the surrounding internal tissue of the patient. Scattered light may be collected in order to create an image of the internal tissue of a patient. The light from red, green, and blue (RGB) lasers can be combined to produce white light that is directed into the illumination single mode optical fiber at the proximal end of the system. This light is conveyed through the system as described herein, and some of the light is scattered by the patient's internal tissue and directed back through the system, also as described above. The light travels through the collection optical fibers from the distal end of the system to the proximal end of the system, where it is then separated into RGB light. The intensity of each color of the light is then measured by optical detectors (not shown in this Figure) and used to create an image of the patient's internal tissue in full color. Alternatively, monochromatic light can be used to produce a single color image, or the white light scattered from the tissue can be detected without dividing it into its RGB color components, to produce a grayscale image of the internal tissue.

In the case of fluorescence imaging, the light from a source that is directed into illumination single mode optical fiber 98 at the proximal end of the system, i.e., the excitation light, is monochromatic and of a wavelength selected to cause a particular type of tissue, such as cancerous tissue, to fluoresce, emitting fluorescent light. The fluorescent light from any possible cancerous tissue, or other tissue of interest, is of a longer wavelength than the monochromatic illumination light that is input into the system to excite the fluorescence. This monochromatic light that is directed into the system at the proximal end, travels through the system and exits the distal end of the system as light 136. Some of the fluorescent light from the patient's internal tissue scatters back into the collection optical fibers 70 and is conveyed through them to the proximal end of the system. An emission filter, which attenuates all of the excitation light, could optionally be disposed in front of the optical detectors at the proximal end of the system, to ensure that only the fluorescent light is detected by the optical detectors.

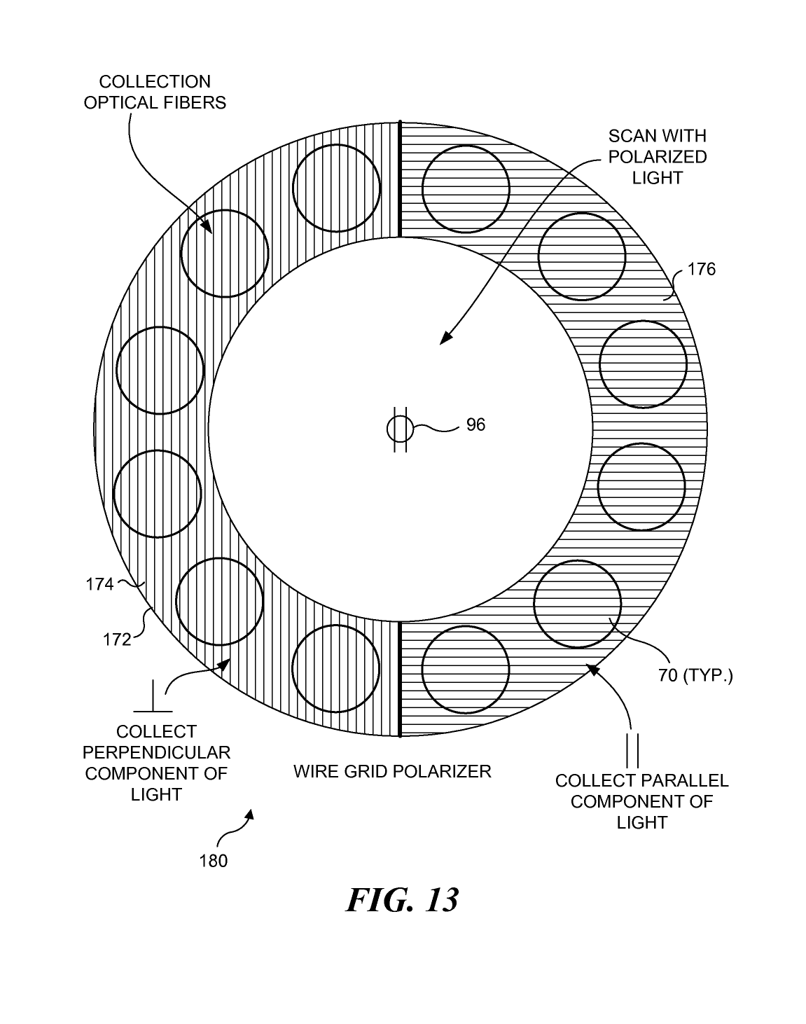

As a further alternative, polarized light can be collected by this system for the imaging of superficial layers of tissue. Light from a source of light (such as a laser—not shown in this Figure) is passed through a polarizing filter and the resulting polarized light is directed into the proximal end of illumination optical fiber 98. In this exemplary embodiment, the illumination optical fiber is a polarization-maintaining single mode optical fiber. The polarized light travels through the illumination optical fiber and exits distal end 96, which is driven to scan in a desired scan pattern. The scanning light is reflected toward internal tissue 132 at the side of the distal end of the system (i.e., at the side of the scope). Some of the light that is reflected or scattered by the internal tissue enters collection optical fibers 70. A polarizer, such as a wire grid polarizer 170 (details of which are shown in FIG. 13 and discussed below) may be positioned over the distal ends of collection optical fibers 70, at the distal end of the system. The polarizer separates the parallel and perpendicular polarized components of the light received from the internal tissue into different groups of collection optical fibers 70, so that intensity of the orientation of polarized light conveyed through a specific optical fiber can be detected by optical detectors (not shown) disposed at the proximal end of the system.

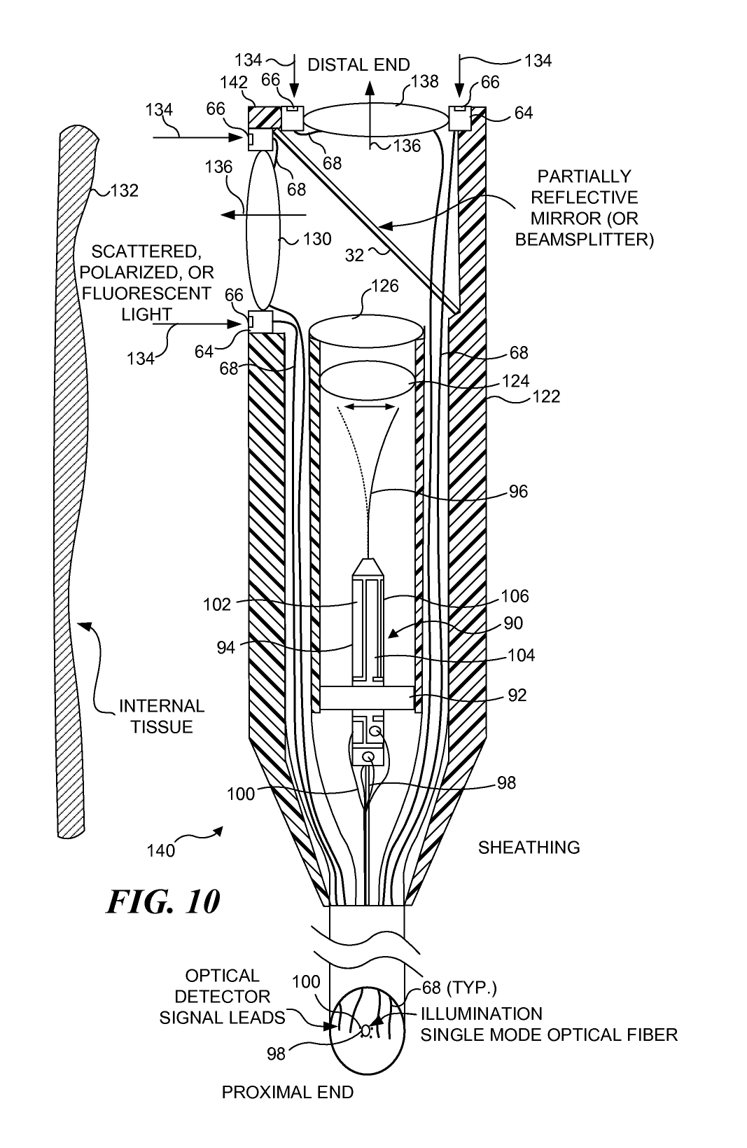

FIG. 10 is a schematic diagram of the part of a side-viewing optical fiber scanning system 140, which can be used internally in a patient, and which includes lenses 124, 126, 130, and 138, and forty-five degree beamsplitter 32. Beamsplitter 32 can be a partially-reflective mirror, a dichroic beamsplitter, or a polarizing beamsplitter. This exemplary embodiment of side-viewing optical fiber scanning system 140 provides both a forward and a side view of a ROI disposed inside the body of a patient, in a manner similar to that illustrated schematically in FIG. 1. Lenses 130 and 138 are encircled by annular detector rings 64 that include optical detectors 66 instead of multimode collection fibers 70. Spaced-apart optical detectors 66 (e.g., photodiodes) receive light 134 from internal tissue 132, both to the side and from forward of the scope, as explained above. The signals produced by the optical detectors are conveyed to the proximal end of the scanning system through detector signal leads 68, where they can be used to produce images of the internal site. This system could be used to collect scattered light, polarized light, or fluorescent light from the surrounding internal tissue of the patient, just as described in connection with system 120 of FIG. 9. All other reference numbers relate to components discussed above in connection with the exemplary embodiment of FIG. 9 and exemplary scanning mechanism 90 of FIG. 7A.

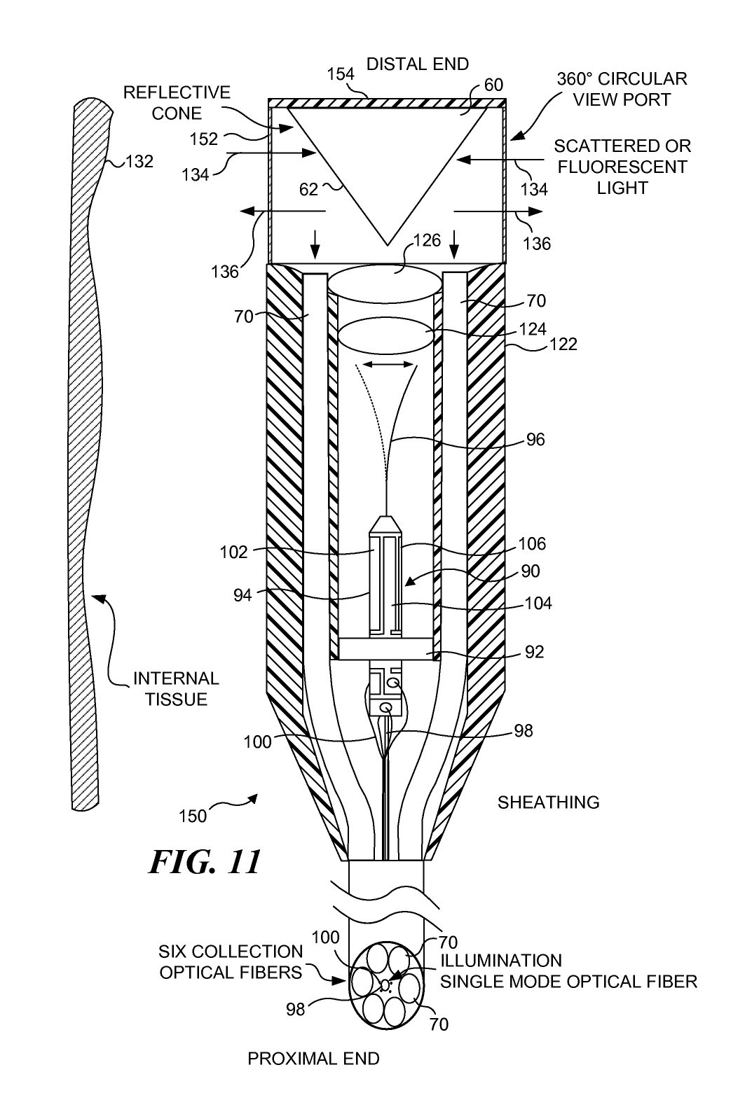

FIG. 11 is a schematic diagram of the distal part of another exemplary embodiment of a side-viewing optical fiber scanning system 150, which can also be positioned internally in a patient. System 150 includes axially-symmetric reflective conical surface 62 (as illustrated in FIG. 5). The conical reflective surface provides a 360° view, but also creates significant image distortion and astigmatism. Therefore, an accurate image of the tissue is not readily created using this conical reflective surface. However, side-viewing optical fiber scanning system 150 is able to accurately distinguish such general tissue conditions as color and fluorescence at an axial position of the scope within a body lumen or other desired site within a patient's body. The other components used in system 150 are generally the same as those shown for side-viewing optical fiber scanning system 120 in FIG. 9, and the description of the components and their functionality provided above is equally applicable to optical fiber scanning system 150.

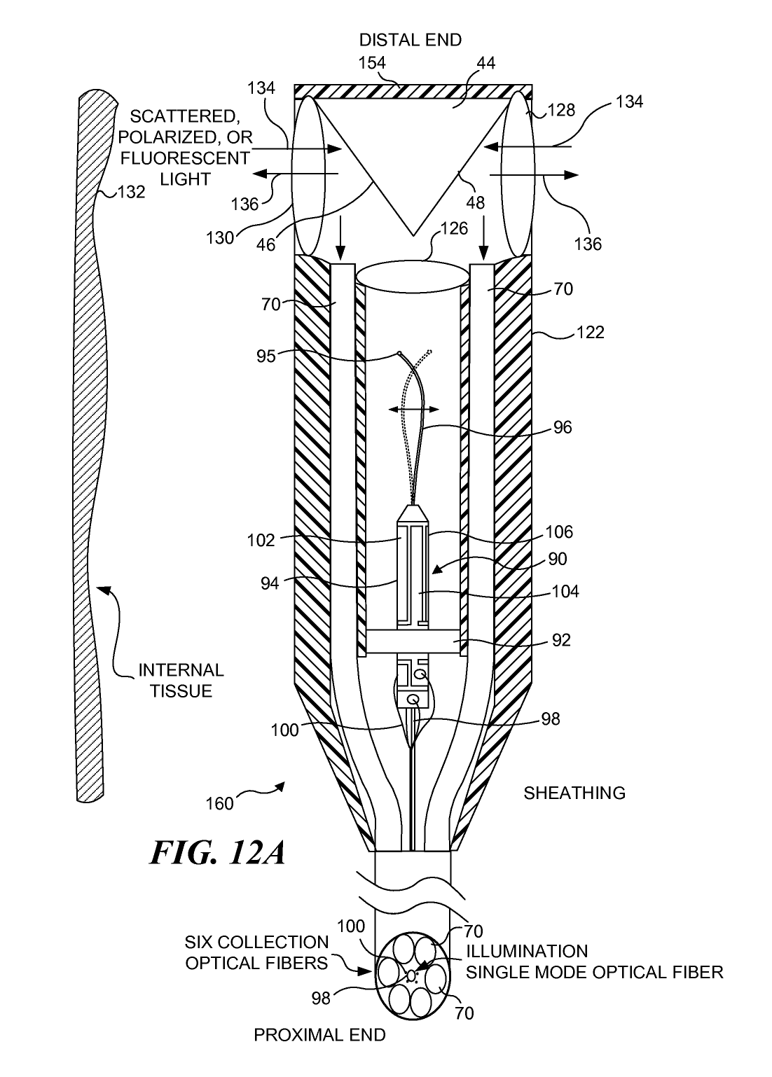

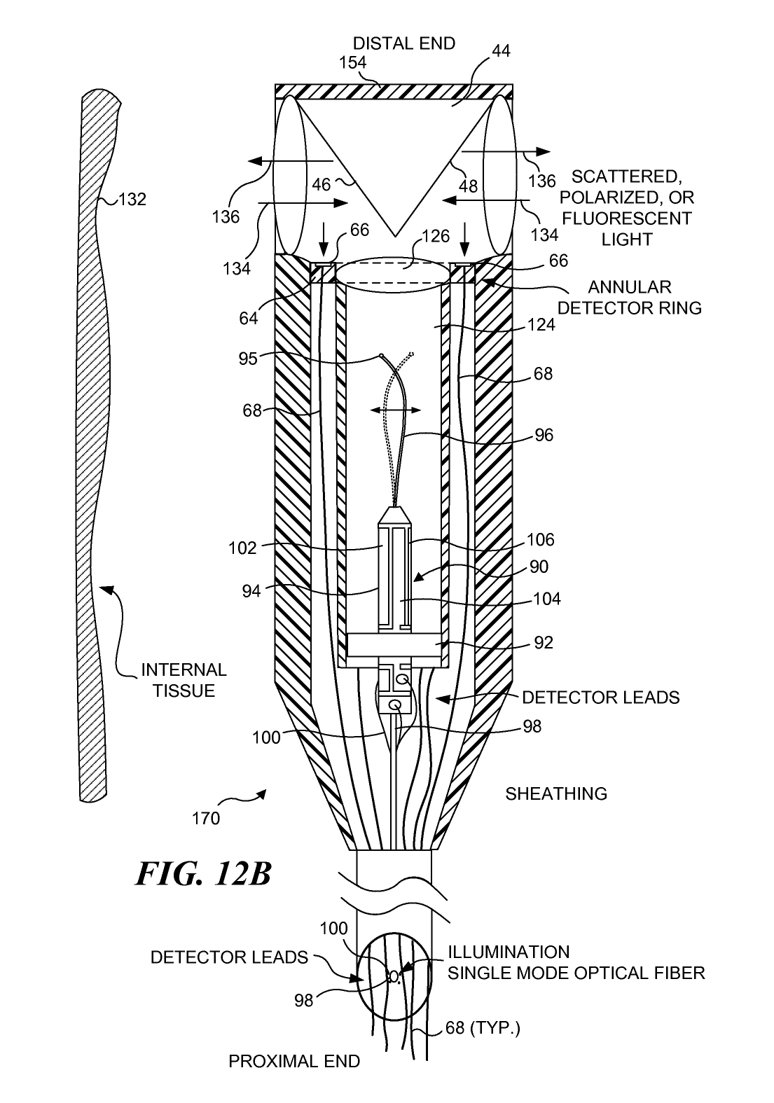

FIG. 12A is a schematic diagram of a distal part of an exemplary optical fiber scanning system 160, which can be used internally in a patient. (Note that the Figure is not drawn to scale and is much smaller in all dimensions—particularly in regard to the diameter of the illustrated scope.) This embodiment includes exemplary scanning mechanism 90, but operating in a manner that is generally similar to that of scanning mechanism 188, to produce a second mode resonance, as illustrated FIG. 7B. Also included in optical fiber scanning system 160 are lenses 126, 128, and 130, and reflective surfaces 46 and 48 (which are shown in FIG. 4A and discussed above) for viewing opposite sides, all supported in sheathing 122. Light from the proximal end of the system is directed through illumination single mode optical fiber 98 and travels in the optical fiber to the distal end of the system, which can be advanced to a desired internal site within a patient's body. Distal end 96 of the single mode optical fiber (but now fused to a microlens 95) is driven to vibrate so as to achieve a desired scan pattern, e.g., a spiral pattern, by tube 94 of piezoelectric material, which is energized by electrical signals applied to its quadrant electrodes, as discussed above (in connection with FIGS. 7A and 7B). Fused at the tip of the single mode cantilever 96 is microlens 95, which produces a semi-collimated beam of illumination that strikes lens 126, as explained above in connection with microlens 196 (shown in FIG. 7B). The light exiting the moving distal end of the optical fiber through microlens 95 travels through lens 126 and is reflected outwardly through lenses 128 and 130 by reflective surfaces 48 and 46, respectively. The supporting structure for these reflective surfaces may be triangular as shown in this exemplary embodiment, having only two reflective surfaces 46 and 48, as also illustrated in FIG. 4A, or alternatively, may comprise pyramidal structure 50 with four reflective surfaces 52, 54, 56, and 58, as discussed above in connection with FIG. 4B. A support structure 140 holds the reflective surfaces and lenses 128 and 130 in place on the distal end of sheath 122. As a further alternative, the conical surface shown in FIG. 5 may be used instead of planar reflective surfaces, as discussed in connection with the exemplary embodiment of side-viewing optical fiber scanning system 150 illustrated in FIG. 11.

Light 136 exiting from the sides of the distal end of the system in FIG. 12A impacts internal tissue 132 of the patient, which might be disposed, for example, at the side of a body lumen. Some of the light may then be scattered from this internal tissue back into the system as scattered light 134, where it travels back through lens 130 and reflects from the reflective surface. Some of the light backscattered or reflected by the tissue is collected by six collection optical fibers 70, the distal ends of which are disposed at spaced-apart locations around lens 126. The light received from the internal tissue is conveyed along the collection fibers from the distal end of the system to its proximal end. Sheathing 122 supports the entire assembly at the distal end of the side-viewing optical fiber scanning system and facilitates its insertion into a lumen or other internal site of the patient.

FIG. 12B is a schematic diagram of the distal part of an exemplary side-viewing optical fiber scanning system 170, which can be used internally in the body of a patient. This system is similar to side-viewing optical fiber scanning system 160 in FIG. 12A, except that the distal end of the system uses annular detector ring 64 with optical detectors 66 (also shown in FIG. 6A) instead of multimode collection fibers 70 to receive light 134 from internal tissue 132. Light is directed through the system from the proximal end to the distal end, as described above for FIG. 12A, and some of the light is scattered from the patient's internal tissue and travels back into the distal end of the system, also as described above. Many of the components of optical fiber scanning system 170 in FIG. 12B are thus identical to those of optical fiber scanning system 160 in FIG. 12A. However, instead of the light from the tissue traveling through collection optical fibers 70 after the light is reflected from reflective surfaces 46 or 48, the light impinges onto optical detectors 66, which are spaced apart around the distal surface of annular detector ring 64, as discussed above in connection with FIGS. 6A and 10. Detector leads 68, which are coupled to the optical detectors, deliver the signal produced by the optical detectors in the annular detector ring at the distal end of the system, to the proximal end of the system, where the information is used to construct an image of the patient's internal tissue. The side-viewing optical fiber scanning system can be used to collect scattered light, polarized light, or fluorescent light from the surrounding internal tissue of the patient, as described for the exemplary embodiment of FIG. 9. In the case of detecting fluorescent light, a suitable emission filter (not shown) is placed over the optical detectors at the distal end of the system, in order to attenuate the wavelength of the excitation light and enable the optical detectors to detect only the fluorescent light, which is readily transmitted through the filter. In a similar fashion, a polarizer is positioned over the optical detectors at the distal end of the side-viewing optical fiber scanning system, in order to detect only polarized light.

A polarizer, such as wire grid polarizer 180, which is shown in FIG. 13, can be used for detecting polarized light and to separate the different orientations of linear polarization. This exemplary embodiment of polarizer 180 is in the form of an annulus 172 with one half of the annular polarizer including a grid with wires 174 oriented perpendicularly to a grid with wires 176 disposed in the other half. If this annular polarizer is positioned over the distal ends of collection optical fibers 70 (or over optical detectors 66) at the distal end of a side-viewing optical fiber scanning system, then half of the collection optical fibers (or optical detectors) will collect one orientation of linear polarization, and the other half of the collection optical fibers will collect light with a linear polarization having an orientation that is perpendicular thereto. Other types of polarizers may be used that serve the same function, e.g., separate optical polarizers can be fitted over the distal ends of each collection optical fiber (or optical detector), with the orientation of the polarization oriented differently for one half of the collection optical fibers (or optical detectors) than for the other half

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

1. A side-viewing scope for imaging a region inside a body of a patient, comprising:
(a) an optical fiber extending between a proximal end and a distal end, the proximal end of the optical fiber being configured to couple to an external light source to receive light produced by the external light source and to convey the light toward the distal end of the optical fiber for use in illuminating a region disposed adjacent to the distal end of the optical fiber;
(b) a scanning device that is disposed at the distal end of the optical fiber and coupled thereto, the scanning device having a free end from which light conveyed through the optical fiber is emitted in a first direction, said scanning device only conveying the light that is used to illuminate;
(c) an actuator for providing a driving force to move the free end of the scanning device in a desired pattern;
(d) a reflective surface disposed adjacent to the free end of the scanning device, the reflective surface reflecting at least a portion of the light emitted from the free end in a second direction that is generally transverse to the first direction, so that at least the portion of the light reflected from the reflective surface is directed towards a side of the scope; and
(e) at least one light detector for detecting light from a region disposed at a side of the scope illuminated by the light reflected from the reflective surface, the at least one light detector producing a signal that is usable to produce an image of the region.

2. The side-viewing scope of claim 1, wherein the reflective surface is selected from the group consisting of:
(a) a mirror that reflects the light emitted by the scanning device in the second direction;
(b) a triangular element having two opposite faces that are reflective and reflect the light emitted by the scanning device in opposite directions, either of the opposite directions comprising the second direction and the other of the opposite directions comprising a third direction;
(c) a cone having a reflective surface;
(d) a pyramidal element having more than two faces that are reflective, each reflecting light emitted by the scanning device in a different direction towards the side of the scope;
(e) a partially-reflective beamsplitter that reflects a portion of the light emitted by the scanning device towards the side of the scope and transmits a remainder of the light;
(f) a dichroic beamsplitter that reflects some wavelengths of the light emitted by the scanning device and transmits other wavelengths; and
(g) a polarizing beamsplitter that reflects linearly polarized light that is polarized in a first direction and transmits linearly polarized light that is polarized in a second direction that is orthogonal to the first direction.

3. The side-viewing scope of claim 2, wherein the partially-reflective beamsplitter transmits a remaining portion of the light emitted by the scanning device towards the distal end of the scope to illuminate another region disposed forward of and proximate to the distal end of the scope, enabling forward viewing by the scope.

4. The side-viewing scope of claim 1, further comprising at least one collection optical fiber having a proximal end and a distal end, wherein the reflective surface also reflects light received from the side of the scope back into the distal end of the at least one collection optical fiber for transmission toward the proximal end of the at least one collection optical fiber.

5. The side-viewing scope of claim 4, wherein the proximal end of each collection optical fiber is configured to couple to a corresponding light detector that detects at least one specific type of light, wherein the specific type of light detected is selected from the group consisting of:
(a) parallel polarized light;
(b) perpendicularly polarized light;
(c) scattered light that has been scattered from tissue;
(d) fluorescent light emitted by tissue; and
(e) the light from the tissue that has been filtered.

6. The side-viewing scope of claim 4, wherein the light that is reflected by the reflective surface towards the side of the scope is polarized.

7. The side-viewing scope of claim 1, wherein the at least one light detector is disposed adjacent to the distal end of the optical fiber, for receiving light from tissue disposed at the side of the scope, the signal produced by the at least one light detector corresponding to an intensity of the light that is received.

8. The side-viewing scope of claim 7, further comprising electrical leads that have a distal end and a proximal end, the distal end of the electrical leads being connected to the at least one light detector for conveying each signal produced thereby to the proximal end of the leads, for coupling to a processing device.

9. The side-viewing scope of claim 1, wherein the actuator applies a driving force to the free end of the scanning device causing the free end to move at about its resonant frequency.

10. The side-viewing scope of claim 1, wherein the actuator causes the scanning device to move in the desired pattern to implement one of:
(a) a linear scan;
(b) a raster scan;
(c) a sinusoidal scan;
(d) a toroidal scan;
(e) a spiral scan; and
(f) a propeller scan.

11. A side-viewing scope for use in scanning a region within a patient's body, comprising:
(a) a flexible optical fiber having a proximal end and a distal end, the proximal end being configured to couple to a light source so that light produced by the light source is conveyed through the optical fiber to the distal end of the optical fiber;
(b) a resonant scanning device disposed at the distal end of the optical fiber to receive the light conveyed through the optical fiber, the resonant scanning device being driven to move in a desired scan pattern at about a resonant frequency of the optical fiber, while emitting the light;
(c) a reflector disposed distally of the resonant scanning device to receive the light emitted by the resonant scanning device and configured to reflect at least a portion of the light received towards a side of the scope for scanning a region disposed at the side; and
(d) at least one collection optical fiber having a proximal end and a distal end, the distal end being disposed to receive light from the region, conveying the light through the at least one collection optical fiber to the proximal end of the at least one collection optical fiber for processing.

12. The side-viewing scope of claim 11, wherein the reflector reflects all of the light emitted from the resonant scanning device radially outward in a plane generally transverse to a longitudinal axis of the side-viewing scope.

13. The side-viewing scope of claim 12, wherein the reflector includes a generally conical reflective surface, further comprising a window in the side-viewing scope that is disposed circumferentially around the conical reflective surface, so that the light reflected outwardly therefrom passes through the window.

14. The side-viewing scope of claim 11, wherein the reflector includes a plurality of reflective surfaces that are oriented at an acute angle relative to a longitudinal axis of the side-viewing scope.

15. The side-viewing scope of claim 14, wherein each of the plurality of reflective surfaces comprises either a triangular surface or a pyramidal surface.

16. The side-viewing scope of claim 11, wherein the reflector comprises a beamsplitter that reflects the portion of the light emitted by the resonant scanner to the side and transmits a remainder of the light forwardly of the side-viewing scope.

17. The side-viewing scope of claim 11, wherein the proximal end of each collection optical fiber is coupled to a detector, the detector producing a signal indicative of an intensity of the light conveyed through the collection optical fiber.

18. The side-viewing scope of claim 17, wherein the signal produced by the detector that receives the light conveyed through the collection optical fiber is indicative of the intensity of at least one type of light selected from the group consisting of:
(a) perpendicularly polarized light;
(b) parallel polarized light;
(c) scattered light produced by light scattering from tissue in the region;
(d) fluorescent light produced by tissue in the region fluorescing; and
(e) light from the tissue that has been filtered.

19. The side-viewing scope of claim 11, wherein the resonant scanning device includes an actuator that when energized, produces a force to cause a cantilevered optical fiber to move in the desired pattern.

20. (canceled)

21. A method for imaging a region disposed at a side of a distal end of a scope that is configured to be introduced into a patient's body, comprising the steps of:
(a) introducing the scope into a patient's body;
(b) conveying light from an external source through an optical fiber toward the distal end of the scope;
(c) moving a free end of a scanning device having a fixed end that is coupled to the optical fiber, so that the free end moves in a desired pattern, emitting light directed generally forward of the optical fiber, the free end of the scanning device only emitting light, and the optical fiber coupled to the scanning device not conveying light from the region;
(d) reflecting at least a portion of the light that is emitted from the scanning device in the desired pattern, towards the side of the scope, to illuminate the region; and
(e) receiving light from the region at the side of the scope, said light being used to produce an image of the region disposed at the side of the scope.

22. The method of claim 21, wherein the step of reflecting light emitted from the scanning device comprises one of the steps of:
(a) reflecting at least the portion of the light emitted from the scanning device in two opposite directions, towards opposite sides of the scope;
(b) reflecting at least the portion of the light emitted from the scanning device in a plurality of different directions, towards different areas around the side of the scope;
(c) reflecting at least the portion of the light emitted from the scanning device in a plane that extends around the sides of the scope; and
(d) splitting the light emitted from the scanning device so a portion of the light is reflected towards the side of the scope, while a remainder of the light is transmitted forward of the scope.

23. The method of claim 22, wherein the step of splitting the light emitted from the scanning device further comprises the step of illuminating another region disposed forward of and proximate to the distal end of the scope.

24. The method of claim 23, further comprising the steps of receiving light from the other region that is disposed forward of and proximate to the distal end of the scope and in response to said light, producing an image of the other region.

25. The method of claim 23, further comprising the step of detecting light from at least one of the group consisting of the region, and of the other region, the light that is detected being of a specific type selected from the group consisting of:
(a) parallel polarized light;
(b) perpendicularly polarized light;
(c) scattered light that has been scattered from tissue;
(d) fluorescent light emitted by tissue; and
(e) light from tissue that has been filtered.

26. The method of claim 21, further comprising the step of causing the light that is reflected towards the side of the scope to be polarized.

27. The method of claim 21, wherein the step of moving the free end of the scanning device comprises the step of driving the free end to move in the desired pattern at about its resonant frequency.

28. The method of claim 21, wherein the step of moving the free end of the scanning device comprises the step of driving the free end to move in the desired pattern implements one of:
(a) a linear scan;
(b) a raster scan;
(c) a sinusoidal scan;
(d) a toroidal scan;
(e) a spiral scan; and
(f) a propeller scan.

29. The method of claim 21, further comprising the step of rotating the scope to increase an angular field of view while the scope is imaging inside the patient's body, the step of rotating the scope occurring at least:
(a) while the scope is positioned at a desired site within the patient's body;
(b) while the scope is being introduced into the patient's body; or
(c) at least while the scope is being withdrawn from the patient's body.

30. The method of claim 29, wherein the step of rotating enables imaging of a lumen within the patient's body over a full 360 degrees, for at least a portion of the lumen.
